(19) United States
(12) Patent Application Publication
(10) Pub. No.: US 2006/0167547 A1
(43) Pub. Date: Jul. 27, 2006

(54) Expandable intervertebral fusion implants having hinged sidewalls

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/337,074

(22) Filed: Jan. 23, 2006

Related U.S. Application Data
(60) Provisional application No. 60/645,026, filed on Jan. 21, 2005.

(51) Int. Cl. A61F 2/44

(52) U.S. Cl. 623/17.11

(57) ABSTRACT

An expandable intervertebral implant includes two shell components connected by articulated side walls which allow for expansion of the components between intervertebral bodies. The implant is maintained at a desired height by placing an insert of a selected size between the articulated walls, to prevent the implant from collapsing.

This application claims benefit under 35 USC 119(e) from provisional patent application 60/645026, filed Jan. 21, 2005.

### BACKGROUND OF THE INVENTION

This invention relates to an expandable intervertebral fusion implant. The class of implements to which this invention pertains serve to stabilize adjacent vertebral elements, thereby facilitating the development of a bony union between them and thus long term spinal stability.

Of all animals possessing a backbone, human beings are the only creatures who remain upright for significant periods of time. From an evolutionary standpoint, this erect posture has conferred a number of strategic benefits, not the least of which is freeing the upper limbs for purposes other than locomotion. From an anthropologic standpoint, it is also evident that this unique evolutionary adaptation is a relatively recent change, and as such has not benefitted from natural selection as much as have backbones held in the horizontal attitude. As a result, the stresses acting upon the human backbone (or “vertebral column”) are unique in many senses, and result in a variety of problems or disease states that are peculiar to the human species.

The human vertebral column is essentially a tower of bones held upright by fibrous bands called ligaments and contractile elements called muscles. There are seven bones in the neck or cervical region, twelve in the chest or thoracic region, and five in the low back or lumbar region. There are also five bones in the pelvic or sacral region which are normally fused together and form the back part of the pelvis. This column of bones is critical for protecting the delicate spinal cord and nerves, and for providing structural support for the entire body.

Between the vertebral bones themselves exist soft tissue structures-discs—composed of fibrous tissue and cartilage which are compressible and act as shock absorbers for sudden downward forces on the upright column. More importantly, the discs allow the bones to move independently of each other, as well. Unfortunately, the repetitive forces which act on these intervertebral discs during repetitive day-to-day activities of bending, lifting and twisting cause them to breakdown or degenerate over time.

Presumably because of humans' upright posture, their intervertebral discs have a high propensity to degenerate. Overt trauma, or covert trauma occurring in the course of repetitive activities disproportionately affect the more highly mobile areas of the spine. Disruption of a disc's internal architecture leads to bulging, herniation or protrusion of pieces of the disc and eventual disc space collapse. Resulting mechanical and even chemical irritation of surrounding neural elements (spinal cord and nerves) cause pain, attended by varying degrees of disability. In addition, loss of disc space height relaxes tension on the longitudinal spine ligaments, thereby contributing to varying degrees of spinal instability such as spinal curvature.

The time-honored method of addressing neural irritation and instability resulting from severe disc damage have largely focused on removal of the damaged disc and fusing the adjacent vertebral elements together. Removal of the disc relieves the mechanical and chemical irritation of neural elements, while osseous union (bone knitting) solves the problem of instability.

To achieve these objectives, a pair of rectangular or semi cylindrical shells joined together by a hinged sidewall are used. These shells are mechanically distracted inside an intervertebral space that has been appropriately prepared for fusion.

As these shells are distracted, the hinged side walls extend from a collapsed or near horizontal attitude to an extended or vertical attitude. Once the ideal degree of expansion has occurred, or the hinged component has opened maximally to a completely vertical attitude, a separate insert component is inserted to prevent closing of the hinged sidewalls so as to maintain separation of the component shells and appropriate expansion of the entire construct.

The expanded construct is then packed with bone or material which can promote osseous union.

The present invention not only provides an expandable intervertebral fusion implant, but also lends itself readily to use in anterior, lateral and posterior approaches. In addition, one can place inserts of different heights in a single intervertebral space to address lateral differences in disc space height to account for degrees of scoliosis, or lateral spinal curvature.

The rectangular or cylindrical implant is split horizontally so that the cranial (upper) and caudal (lower) shells that contact the vertebral bones above and below can be distracted, or spread apart, by a screw-type or plier type installation tool, until optimal distraction of the vertebral elements and appropriate tension on the ligamentous structures is achieved. Once this occurs, an internal insert is inserted to prevent collapse of the hinged sidewalls thereby forming a stable construct that remains in its expanded state ready to be filled with bone or fusion material.

The advantages provided by this invention include a design that is simple to manufacture, allows for an expandable function which lends itself to use in minimally invasive or microsurgical approaches, and utilizes a structural design which permits the used of a variety of construction materials (e.g. titanium, carbon fiber, graphite, PEEK, nitinol, plastics, composites, etc.).

### BRIEF DESCRIPTION OF THE DRAWINGS

FIG. 1 is a perspective view of an intervertebral fusion implant embodying the invention, in a collapsed configuration;

FIG. 2 is a similar view, showing the implant in a distended configuration;

FIG. 3 shows an insert prior to placement within the implant;

FIG. 4 shows the insert being placed within the implant;

FIG. 5 shows the insert fully within the implant;

FIG. 6 is an end view of the implant, shown filled with bone growth material between adjacent intervertebral bodies;

FIG. 7 is a side view thereof;

FIGS. 8-12 show corresponding views of a second embodiment of the invention;

FIGS. 13 and 14 are rear and side views, respectively, showing a spinal site which has been prepared for insertion of implants according to this invention;

FIGS. 15 and 16 are similar views, showing a collapsed implant, of the type shown in FIGS. 8-12, being inserted into the site;

FIGS. 17 and 18 show the implants having been expanded; and

FIGS. 19 and 20 show inserts placed within the implants to maintain them in their expanded state.

### DESCRIPTION OF THE PREFERRED EMBODIMENTS

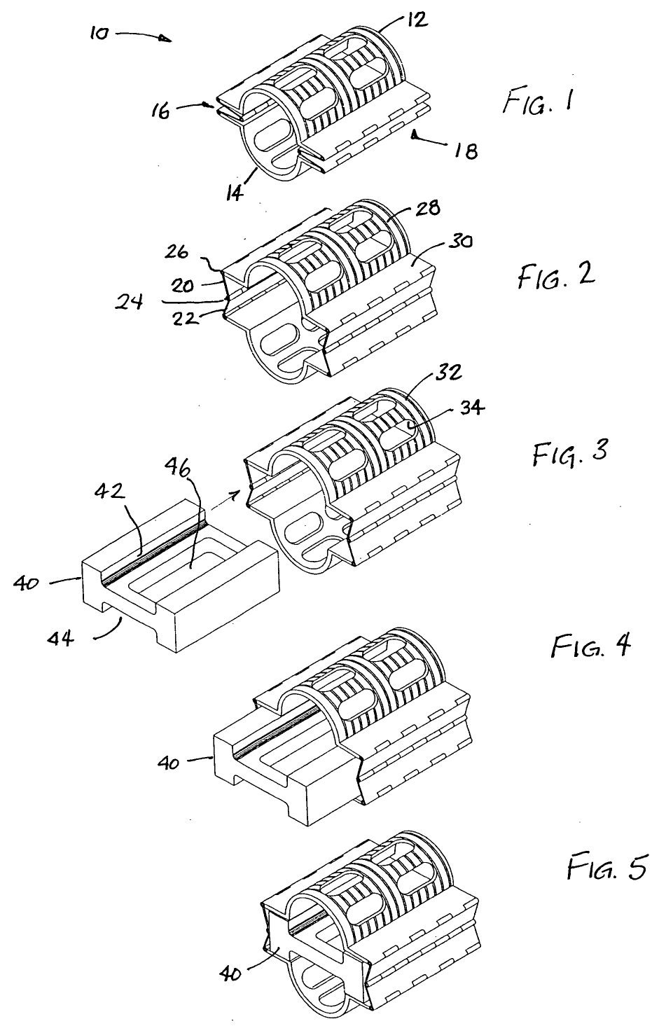

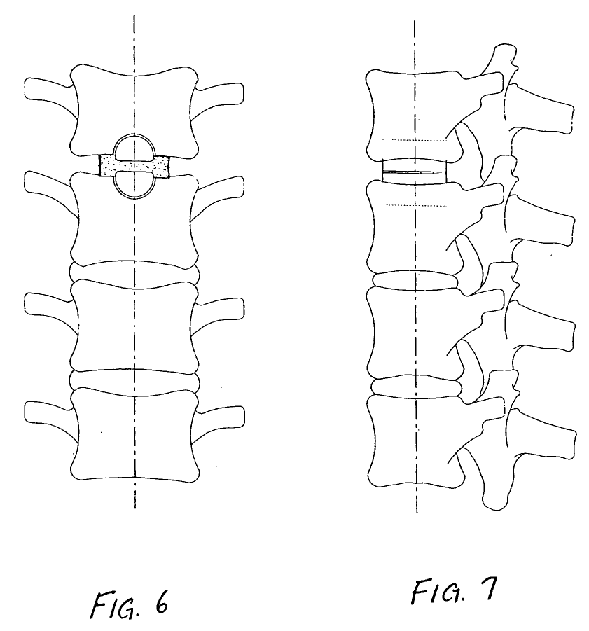

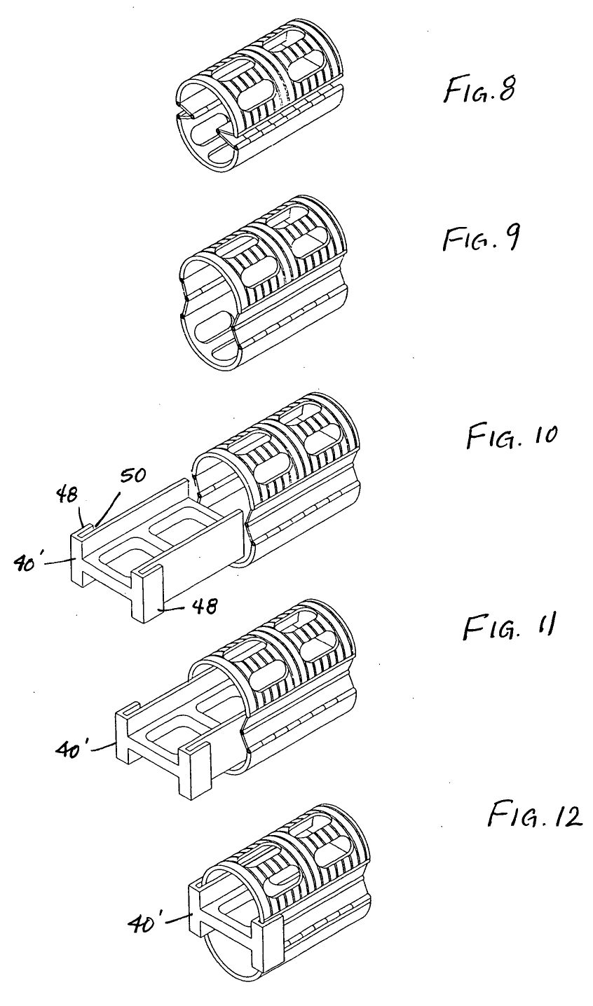

The outer surfaces of the rectangular or semicylindrical shells preferably have points or ridges on them which dig into the adjacent vertebral body to prevent shifting of the expanded implant. Windows are provided in each of the shells to encourage growth of bony material which immobilizes the implant. An expandable intervertebral fusion implant 10 embodying the invention includes a pair of shells 12,14 which are adjustably distanced from each other, while being maintained parallel, by a pair of articulated side walls 16,18. Each side wall has a pair of hinged leaves 20,22. The leaves are interconnected along their inner edges by a hinge pin 24. The outer edges of the leaves are connected to the respective shells by hinge pins 26. The hinge pins 26 are shown at the outer edges of each of the shells in FIGS. 1-5, but they might be situated inboard of the outer edges.

Each of the shells shown in FIGS. 1-5 has a semicylindrical portion 28 and a pair of wings 30 astride the semicylindrical portion. The hinge pins 26 extend through hinge structure formed at the outer edge of each wing.

The outer surfaces of the rectangular or semicylindrical shells preferably have or circumferential ridges 32 or points (not shown) on them which dig into the adjacent vertebral body to prevent shifting of the expanded implant. Windows 34 are provided in each of the shells to encourage growth of bony material into the windows, which immobilizes the implant.

On each implant, the hinged side walls fold inward (or outward, if desired) to a collapsed configuration to minimize the overall lateral dimension of the implant, thereby making it useful in minimally invasive or microsurgical laminotomy approaches.

After the implant has been properly situated in the surgical site (FIGS. 6 and 7), the shells are distracted with a suitable tool, whereupon the hinged sidewalls unfold passively to a more vertical attitude.

Once the proper degree of expansion has been achieved—as determined by the surgeon—in order to tauten intervertebral ligaments, an insert 40 of a desired width is placed between the shells to prevent the sidewalls from collapsing, thereby maintaining the appropriate expansion of the implant. The insert has grooves 42,44 top and bottom, and a central aperture 46, to facilitate the placement of bone growth material and to encourage such material to immobilize the insert. FIGS. 6 and 7 show the implant in place.

By changing the size of the intervening insert, varying degrees of expansion of the implant can be maintained. An assortment of inserts of different sizes may be provided with the implant, to facilitate this adjustment.

FIGS. 8-14 show a second embodiment of the invention, where the shells lack the wings provided in the first embodiment. This reduces the width of the implant, making it more suitable for procedures, such as that illustrated in FIGS. 13-20, in which two implants, quite possibly of different installation heights, are inserted between the same pair of intervertebral bodies. This enables the surgeon to realign a spine having improper curvature.

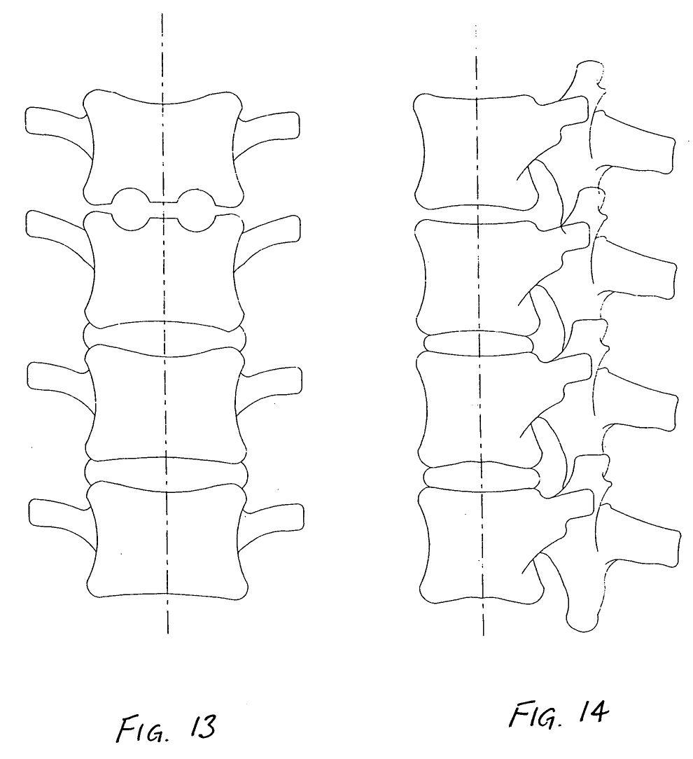

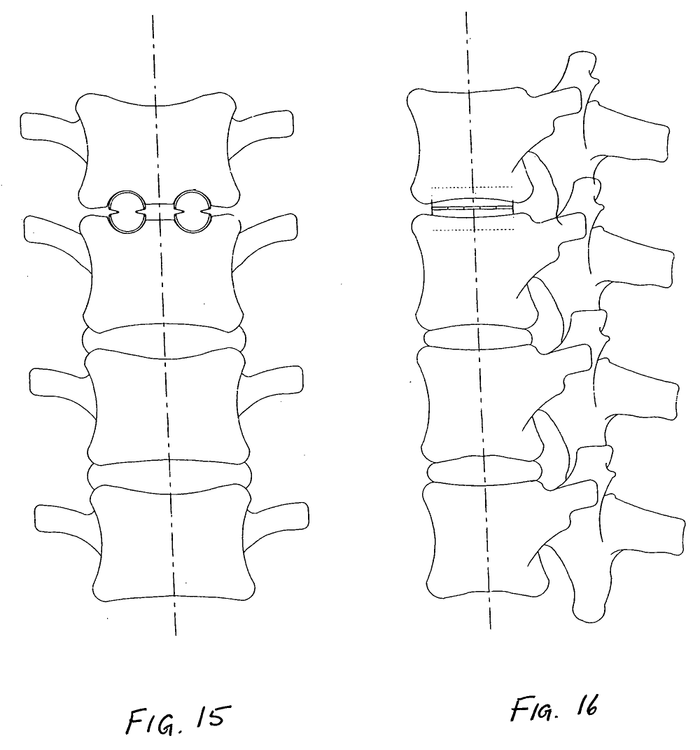

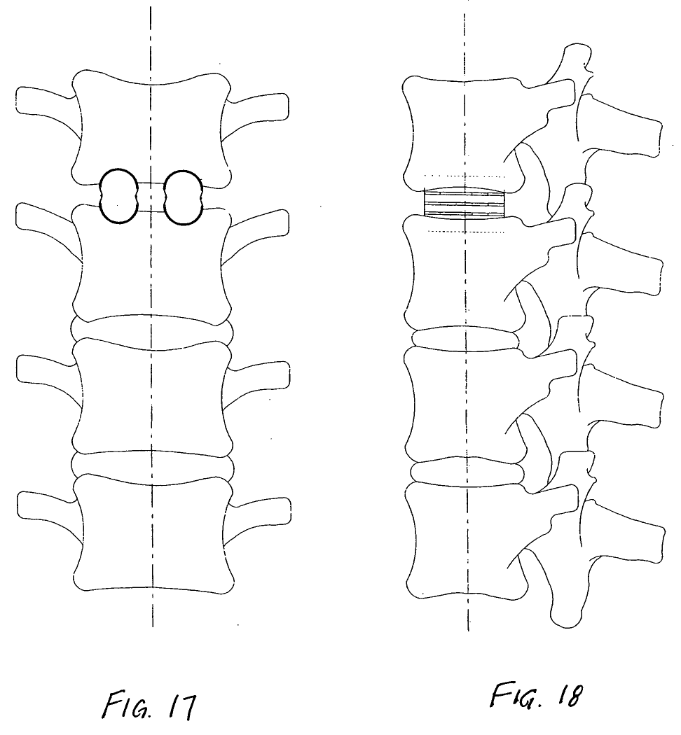

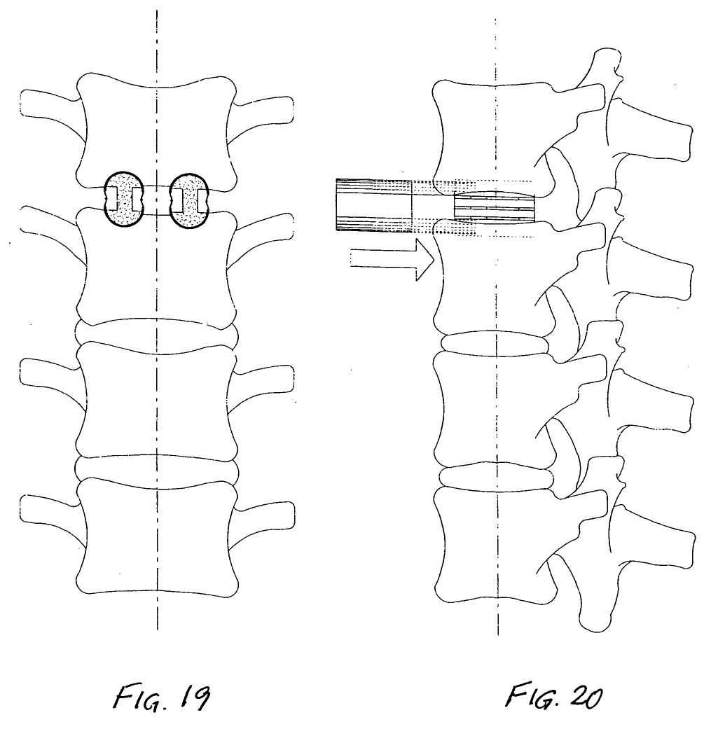

FIGS. 13-20 show the steps of site preparation (FIGS. 13-14), implant insertion (FIGS. 15-16), implant expansion (FIGS. 17-18) and insert placement (FIGS. 19-20).

The insert 40′ shown in FIGS. 10-12 differs from the insert 40 described previously in that is has a pair of fingers 48 which define grooves 50 that receive the side walls when the insert is placed. The fingers hook over the side walls and prevent them from folding outward, providing more secure engagement with the implant.

Once the implant has been suitably deployed and locked in the expanded state by the intervening insert, the implant can be packed with bone or similar osseous fusion material so that a stable arthrodesis or fusion can occur.

Inasmuch as the invention is subject to many changes and variations in detail, it is intended the at the foregoing should be regarded merely as exemplary of the invention defined by the claims below.

I claim:

1. An expandable intervertebral fusion implant comprising a pair of rectangular or semi cylindrical shells joined by at least two articulated side walls, each comprising at least two leaves which can hinge with respect to one another to allow the implant to be collapsed prior to implantation and then expanded once implanted.

2. The invention of claim 1, wherein the shells and the side walls are made of a material selected from the group consisting of titanium alloy, steel, nitinol, carbon fiber, PEEK, graphite or plastics, and combinations thereof.

3. The invention of claim 1, further comprising an insert sized for placement between the hinged side walls to limit maintain the implant in its expanded configuration.

4. The invention of claim 3, wherein the insert has finger portions adapted to hook over the side walls and prevent them from collapsing by folding in an outward direction.

5. An expandable intervertebral fusion implant kit comprising
a implant having a pair of shells joined by at least two articulated side walls, each side wall comprising at least two leaves which can hinge with respect to one another to allow the implant to be collapsed prior to implantation and then expanded once implanted, and
an assortment of inserts of different sizes, any of which can be placed within the implant, between the side walls, to maintain the implant at a desired height.
